(19) United States
(12) Patent Application Publication
(10) Pub. No.: US 2010/0042078 A1
(43) Pub. Date: Feb. 18, 2010

(54) INTRA-BODY-CAVITY INSERTION INSTRUMENT GUIDE AND INTRA-BODY-CAVITY INSERTION INSTRUMENT GUIDE SYSTEM

(75) Inventor: Yuta OKADA, Hachioji-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/603,876

(22) Filed: Oct. 22, 2009

Related U.S. Application Data
(63) Continuation of application No. PCT/JP2008/058945, filed on May 15, 2008.

(30) Foreign Application Priority Data
May 16, 2007 (JP) 2007-130896

(51) Int. Cl. A61B 17/94

(52) U.S. Cl. 606/1

(57) ABSTRACT

An intra-body-cavity insertion instrument guide includes an insertion section that is inserted into a body cavity, and a guide section that is formed at the insertion section and guides an intra-body-cavity insertion instrument for obtaining a space for operating on an organ in the body cavity, to a side opposite to an insertion hole into which the insertion section is inserted, in relation to the organ.

### CROSS-REFERENCE TO RELATED APPLICATIONS

This is a Continuation Application of PCT Application No. PCT/JP2008/058945, filed May 15, 2008, which was published under PCT Article 21(2) in Japanese.

This application is based upon and claims the benefit of priority from prior Japanese Patent Application No. 2007-130896, filed May 16, 2007, the entire contents of which are incorporated herein by reference.

### BACKGROUND OF THE INVENTION

1. Field of the Invention

The present invention relates to an intra-body-cavity insertion instrument guide and an intra-body-cavity insertion instrument guide system for guiding an intra-body-cavity insertion instrument for obtaining a space in a body cavity to allow an organ in the body cavity to be operated on in a surgical operation.

2. Description of the Related Art

In cardiac surgery, sterna are cut (e.g., median sternotomy) to allow access to the chest cavity. In this case, a retractor is set in the opening made in the chest and separates widely the sterna and organs, to create a large opening. Surgical instruments are deployed through the opening, and cardiac surgery is then carried out.

One of most common forms of cardiac surgery is coronary artery bypass grafting (CABG). In CABG, occlusion in one or plural coronary arteries is bypassed by connecting a graft to a coronary artery on the downstream side of the occlusion. The technique for connecting a graft to a coronary artery is known as an anastomosis. As a graft, for example, a mammary artery which is cut from the chest wall is used. In that case, an upstream end of the mammary artery is left intact while the other downstream end of the mammary artery is connected to the coronary artery. Alternatively as a graft, an artery or vein cut from any part of the human body may be used. Also alternatively, a piece of artificial blood vessel may be used as a graft. In that case, an upstream end of the piece grafted is connected to an artery such as an aorta while the other downstream end thereof is connected to the coronary artery. In this manner, occlusion in plural coronary arteries at various positions in the heart, such as in the front, side, or back of the heart, is bypassed by using plural grafts.

Meanwhile, in recent years, as minimally invasive surgery, endoscopic operations have been carried out in the aforementioned CABG, to conduct various procedures by forming a hole in a wall of a human body cavity such as an abdominal wall and by further inserting an endoscope or a surgical instrument into the body cavity through the hole. In that case, a monitor camera is inserted at a position in the body cavity which corresponds to an affected portion appearing on a CT image picked up prior to the operation.

Such an endoscopic surgical operation, for example as disclosed in Patent Document 1, uses a retractor whose plural flat plates are inserted into a body cavity and then open in fan-like fashion, so that organs other than a target organ to be operated on are pressed and excluded, thereby to maintain a view field for a monitor camera.

Patent Document 1: Jpn. Pat. Appln. KOKAI Publication No. 6-154152

### BRIEF SUMMARY OF THE INVENTION

Accordingly, the present invention provides an intra-body-cavity insertion instrument guide and an intra-body-cavity insertion instrument guide system, which are capable separating a target organ existing deep in a body cavity from other organs, to obtain a space for surgery.

According to one aspect of the invention, there is provided an intra-body-cavity insertion instrument guide comprising: an insertion section that is inserted into a body cavity; and a guide section that is formed at the insertion section and guides an intra-body-cavity insertion instrument for obtaining a space for operating on an organ in the body cavity, to a side opposite to an insertion hole into which the insertion section is inserted, in relation to the organ.

According to one other aspect of the invention, there is provided an intra-body-cavity insertion instrument guide system comprising: an intra-body-cavity insertion instrument that obtains, in a body cavity, a space for operating on an organ in the body cavity; and a guide that includes an insertion section inserted into the body cavity, and a guide section that is formed at the insertion section and guides the intra-body-cavity insertion instrument to a side opposite to an insertion hole into which the insertion section is inserted, in relation to the organ.

### BRIEF DESCRIPTION OF THE SEVERAL VIEWS OF THE DRAWING

FIG. 1A is a view illustrating a structure of an intra-body-cavity insertion instrument guide according to the first embodiment of the present invention;

FIG. 1B is a schematic view for describing operation of an intra-body-cavity insertion instrument guide system according to the first embodiment of the invention;

FIG. 1C is a schematic view for describing operation of the intra-body-cavity insertion instrument guide system according to the first embodiment of the invention;

FIG. 2A is a view illustrating a structure of an intra-body-cavity insertion instrument guide system according to the second embodiment of the invention;

FIG. 2B is a view illustrating an intra-body-cavity insertion instrument in the intra-body-cavity insertion instrument guide system according to the second embodiment;

FIG. 3A is a schematic view for describing operation of the intra-body-cavity insertion instrument guide system according to the second embodiment;

FIG. 3B is a schematic view for describing operation of the intra-body-cavity insertion instrument guide system according to the second embodiment;

FIG. 3C is a schematic view for describing operation of the intra-body-cavity insertion instrument guide system according to the second embodiment;

FIG. 3D is a schematic view for describing operation of the intra-body-cavity insertion instrument guide system according to the second embodiment;

FIG. 4 is a view illustrating a structure of an intra-body-cavity insertion instrument guide system according to the third embodiment of the invention;

FIG. 5 is a view illustrating a structure of an intra-body-cavity insertion instrument guide system according to the fourth embodiment of the invention;

FIG. 6 is a view illustrating an intra-body-cavity insertion instrument in an intra-body-cavity insertion instrument guide system according to the fifth embodiment of the invention;

FIG. 7A is a schematic view for describing operation of the intra-body-cavity insertion instrument guide system according to the fifth embodiment;

FIG. 7B is a schematic view for describing operation of the intra-body-cavity insertion instrument guide system according to the fifth embodiment;

FIG. 7C is a schematic view for describing operation of the intra-body-cavity insertion instrument guide system according to the fifth embodiment;

FIG. 7D is a schematic view for describing operation of the intra-body-cavity insertion instrument guide system according to the fifth embodiment; and

FIG. 8 is a view illustrating a modification example of the intra-body-cavity insertion instrument in the intra-body-cavity insertion instrument guide system according to the fifth embodiment.

### DETAILED DESCRIPTION OF THE INVENTION

Hereinafter, best mode for carrying out the present invention will be described with reference to the drawings.

### First Embodiment

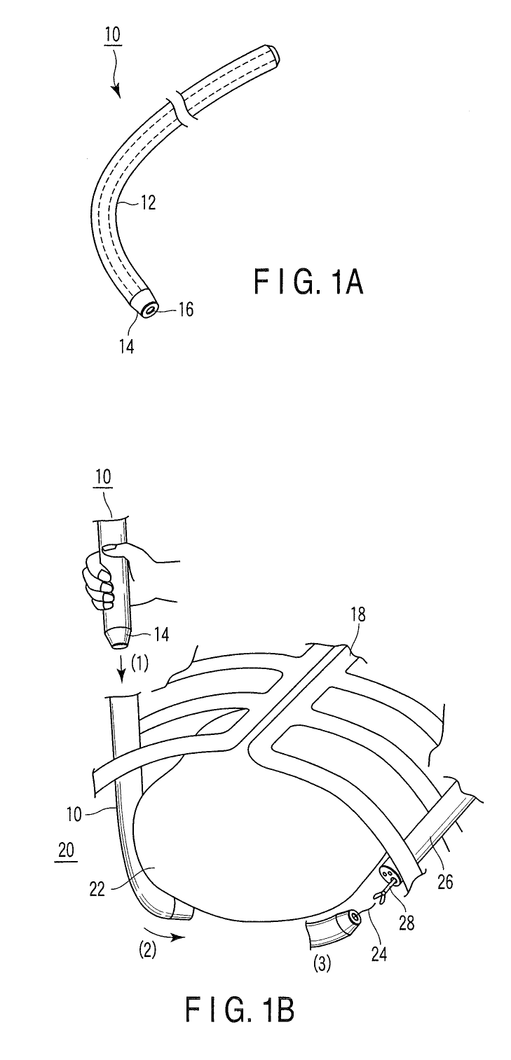

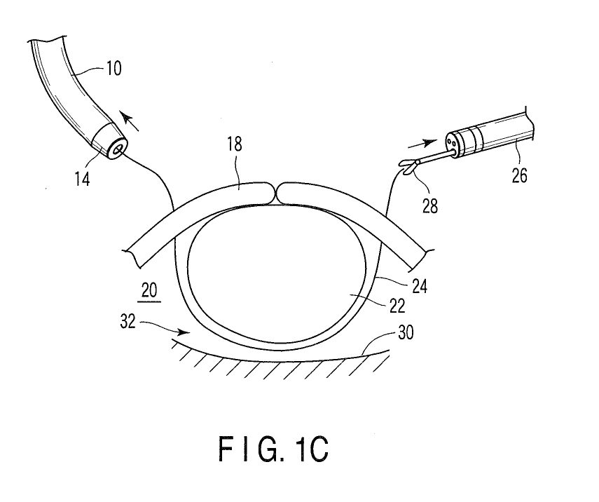

FIG. 1A is a view illustrating a structure of an intra-body-cavity insertion instrument guide 10 according to the first embodiment of the invention.

The intra-body-cavity insertion instrument guide 10 according to the present embodiment includes an insertion section 12 to be inserted into a body cavity. The insertion section 12 is made of a soft tube which is prone to bending. An end of the insertion section 12 is formed as a tapered hard top end 14 in order to improve insertability. A lumen 16 for inserting an intra-body-cavity insertion instrument is formed throughout the whole length of the insertion section 12. The lumen 16 functions as a guide section for guiding the intra-body-cavity insertion instrument toward a side opposite to an insertion hole in which the insertion section 12 is inserted, in relation to an organ.

FIGS. 1B and 1C are views illustrating operation of an intra-body-cavity insertion instrument guide system according to the first embodiment of the invention, which uses the intra-body-cavity insertion instrument guide according to the first embodiment having a structure as described above.

Specifically, as denoted at (1) in FIG. 1B, an operator firstly inserts the intra-body-cavity insertion instrument guide 10 into a body cavity 20 through between ribs 18 from an insertion hole (not illustrated) formed in the body wall, with the hard top end 14 of the intra-body-cavity insertion instrument guide 10 oriented downward in the gravitational direction. The intra-body-cavity insertion instrument guide 10 is then guided along a bottom face of a target organ 22 such as the heart, as denoted at (2) in FIG. 1B, because the intra-body-cavity insertion instrument guide 10 is prone to bending as described previously. Further, as denoted at (3) in FIG. 1B, the hard top end 14 reaches to the side opposite to the insertion hole, and then, a cord 24 as an intra-body-cavity insertion instrument is inserted into the lumen 16 from a rear end of the lumen 16, so as to extend throughout the lumen 16. A top end of the cord 24 protrudes from the hard top end 14. Thereafter, the top end of the cord 24 is grasped by a pair of biopsy forceps 28 extended through a forceps channel included in an endoscope 26 which is inserted into another insertion hole formed in the body wall in a side opposite to the foregoing insertion hole. Kept grasped by the biopsy forceps 28, the endoscope 26 is pulled out of the human body, and the top end of the cord 24 is accordingly pulled out of the human body.

Further, as illustrated in FIG. 1C, the intra-body-cavity insertion instrument guide 10 is also pulled out of the human body, and the cord 24 is pulled from the rear end of the intra-body-cavity insertion instrument guide 10. The cord 24 is thereby left extending along the bottom face of the target organ 22 in the body cavity 20. By then pulling out two ends of the cord 24 (or by pulling out one end of the cord 24 with the other end thereof fixed), the target organ 22 is lifted up against gravity and separated from another organ 30. An operating space 32 for surgery is accordingly obtained between the target organ 22 and the another organ 30.

Once the operating space 32 is obtained in this manner, two ends of the cord 24 outside the human body are then engaged on the ribs 18 or a frame not illustrated, and the obtained operating space 32 can be thereby maintained.

Since the target organ 22 is supported by pinching (i.e., not by cantilevering), rigidity of the intra-body-cavity insertion instrument may be so low that rigidity of such a thin instrument as the cord 24 is satisfactory.

Further, the intra-body-cavity insertion instrument guide 10 can be configured to be thin since the cord 24 is used as an intra-body-cavity insertion instrument. Accordingly, there is an effect that even a small hole is satisfactory for insertion of the intra-body-cavity insertion instrument guide 10.

### Second Embodiment

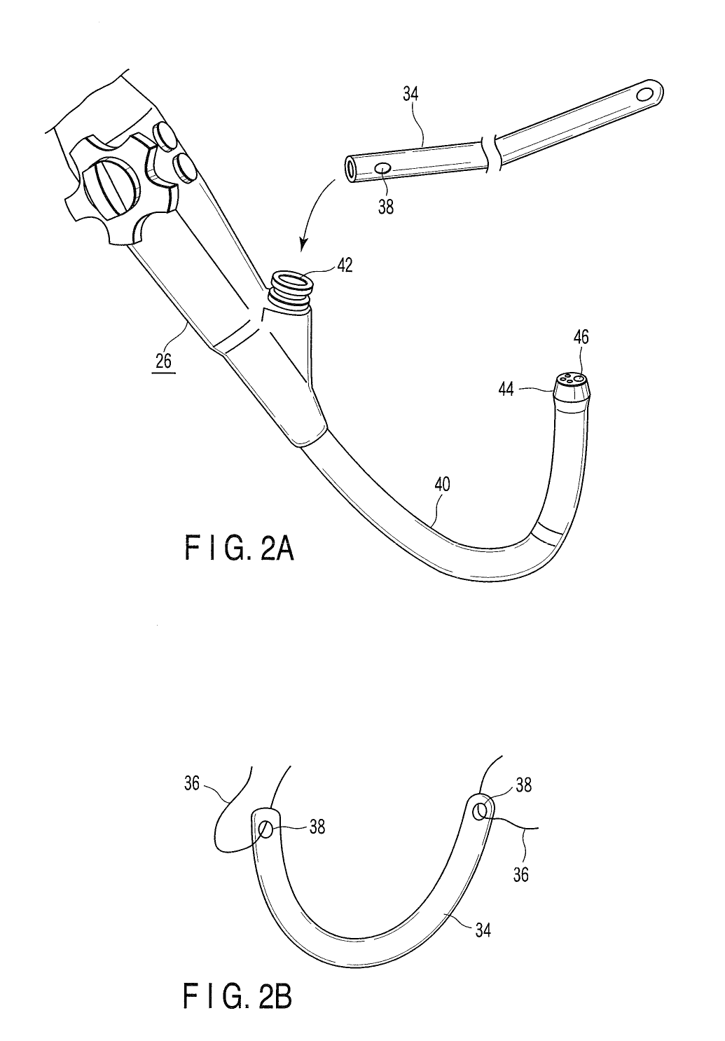

FIG. 2A is a view illustrating a structure of an intra-body-cavity insertion instrument guide system according to the second embodiment of the invention.

In the second embodiment, a PTFE (i.e., Teflon (registered trademark)) tube 34 is used as an intra-body-cavity insertion instrument. Side holes 38 which allow a cord 36, as a pulling member for pulling an organ from inside of a body cavity to outside of the human body, to pass are formed at near two ends of the PTFE tube 34, as illustrated in FIG. 2B.

The intra-body-cavity insertion instrument guide according to the second embodiment of the invention is an endoscope 26 including a forceps channel having a diameter which allows such a PTFE tube 34 to be inserted. The present embodiment will now be described referring to an example of using the endoscope 26 as an intra-body-cavity insertion instrument guide. Needless to say, however, the intra-body-cavity insertion instrument guide may be made of a soft tube which is prone to bending as in the first embodiment.

Specifically, a forceps channel for inserting a pair of biopsy forceps is provided in an insertion section 40 of the endoscope 26, and connects a top end of the insertion section 40 to a section which is positioned outside the human body when the insertion section 40 is inserted into a body cavity 20. The present embodiment uses the forceps channel as a guide section. The PTFE tube 34 is inserted from an inlet port 42 of the forceps channel, extends through the forceps channel, and protrudes from an outlet port 46 of a tapered hard top end 14.

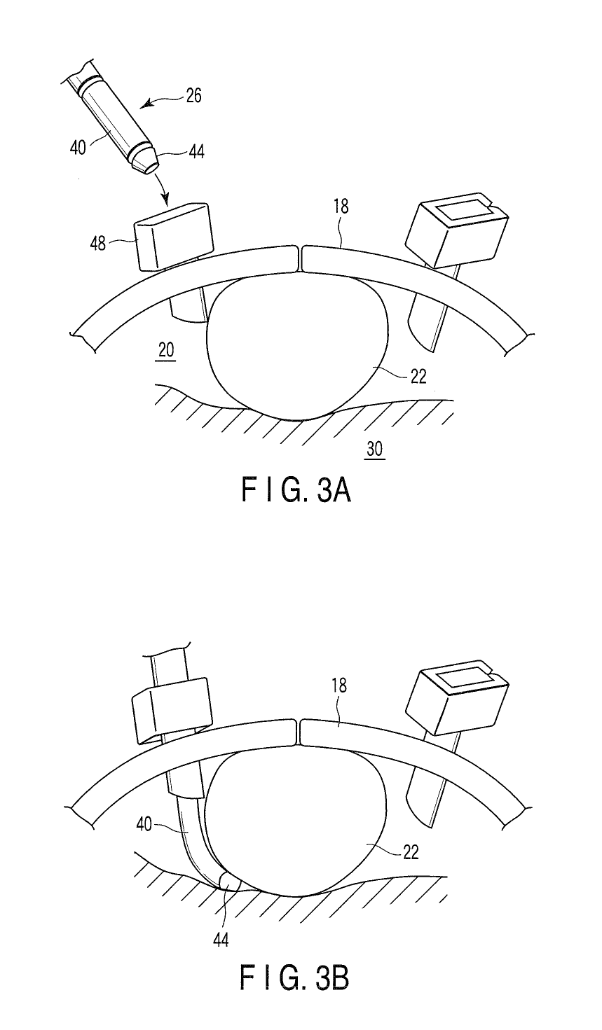

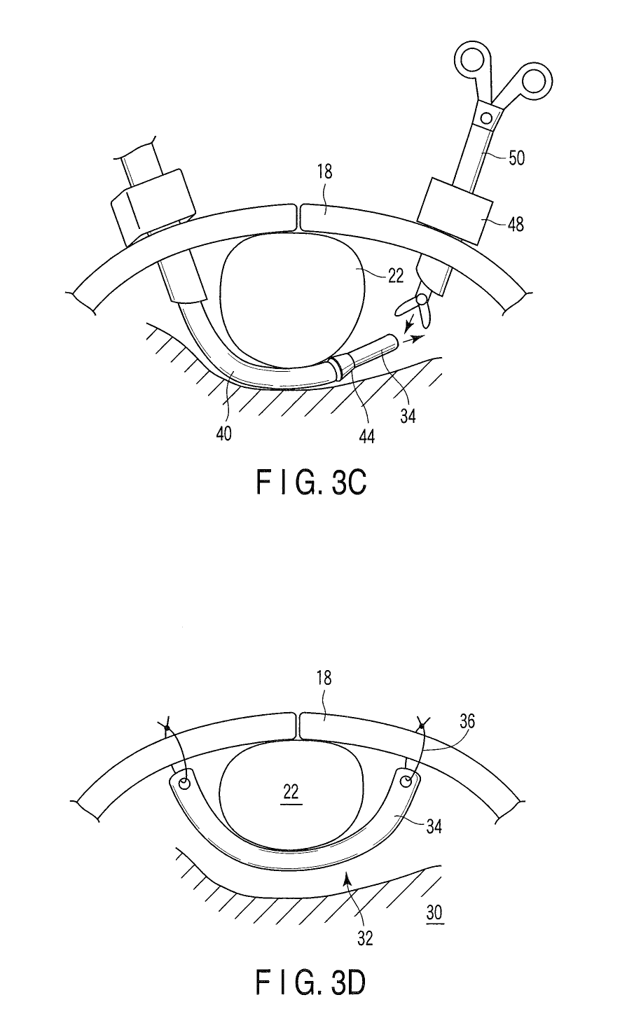

FIGS. 3A, 3B, 3C, and 3D are views illustrating operation of the intra-body-cavity insertion instrument guide system according to the second embodiment.

As illustrated in FIG. 3A, in order to insert and pull out a instrument which is as thick as the endoscope 26 into and from a body cavity 20, a trocar 48 is fixed to an insertion hole formed at a position between ribs 18, and the endoscope 26 is inserted into a body cavity 20 through the trocar 48. Further as illustrated in FIG. 3B, the endoscope 26 is slipped in under the target organ 22, bend along the target organ 22 such as the heart.

As illustrated in FIG. 3C, when a hard top end 44 of the endoscope 26 reaches an opposite side over the target organ 22, the PTFE tube 34 is then inserted into the forceps channel, and a top end of the PTFE tube 34 is made to protrude from the hard top end 14. At this time, the cord 36 is inserted into the side hole 38 in the rear end side of the PTFE tube 34 while the cord 36 is not inserted into the other side hole 38 in the top end side. Further, a pair of forceps 50 is inserted into the body cavity 20 through another trocar 48 fixed to a side opposite to the foregoing trocar 48 in which the endoscope 26 is inserted. The PTFE tube 34 is grasped by the forceps 50 and then pulled out of the human body through the another trocar 48.

Further, the cord 36 is inserted into the side hole 38 in the top end side of the PTFE tube 34 pulled out of the human body, and is then engaged on the ribs 18 or a frame not illustrated. If the endoscope 26 is now pulled out of the human body, the PTFE tube 34 is left remain in the body cavity 20 since the top end of the endoscope 26 is fixed. Hence, through the trocar 48 after the endoscope 26 has been pulled out, the cord 36 inserted into the rear end side or the side hole 38 of the PTFE tube 34 is grasped by the forceps 50, further pulled out of the human body, and then engaged on the ribs 18 or a frame not illustrated. In this manner, the PTFE tube 34 lifts up the bottom face of the target organ 22 against gravity in the body cavity 20, and separates the target organ 22 from another organ 30. A operating space 32 for surgery can accordingly be obtained between the target organ 22 and the another organ 30.

Thus, also according to the second embodiment, the operating space 32 can be obtained by supporting the target organ 22 by pinching (i.e., not by cantilevering).

Further, the second embodiment uses the PTFE tube 34 which has a certain width, as an intra-body-cavity insertion instrument, in place of a thin cord in the first embodiment. Therefore, when the intra-body-cavity insertion instrument is positioned lower in the gravitational direction, the target organ 22 is pressed against the intra-body-cavity insertion instrument due to gravity, so that not only the operating space 32 can be obtained but also movement of the target organ 22 such as pulsation can be restricted.

Since the guided PTFE tube 34 is once left remain in the body cavity 20, the position of the PTFE tube 34 can be finely adjusted by using the forceps 50.

### Third Embodiment

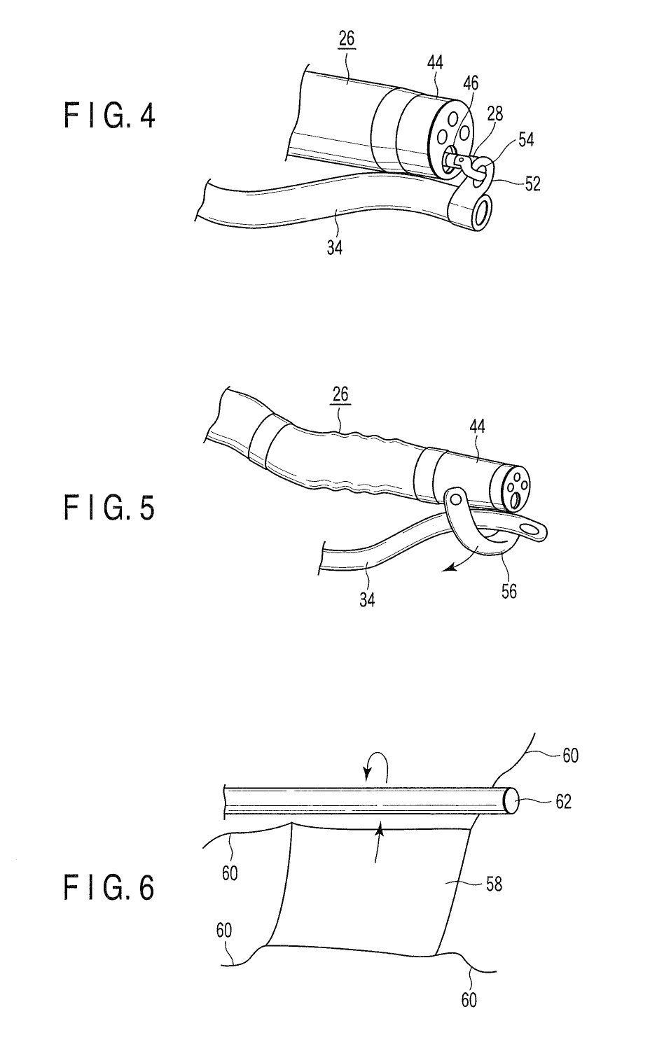

FIG. 4 is a view illustrating a structure of an intra-body-cavity insertion instrument guide system according to the third embodiment of the invention.

In the third embodiment, a PTFE tube 34 as described above is used as an intra-body-cavity insertion instrument, and an engaging member 52 is provided at a top end of the PTFE tube 34. An engaging hole 54 is formed in the engaging member 52.

In case of an endoscope 26 as an intra-body-cavity insertion instrument guide according to the third embodiment of the invention, the engaging hole 54 of the engaging member 52 is grasped by a pair of biopsy forceps 28 which is extended through a forceps channel and made to protrude from an outlet port 46.

Therefore, the PTFE tube 34 can be guided simultaneously together with the endoscope 26 by inserting the endoscope 26 into the body cavity 20 while maintaining the grasp as described above. Further, when the PTFE tube 34 reaches an opposite side over a target organ 22, the grasp by the biopsy forceps 28 is then released, and the endoscope 26 is pulled out of the human body. The PTFE tube 34 is then left remain in the body cavity 20.

Thereafter, two ends of the PTFE tube 34 are engaged on ribs 18 or a frame not illustrated by the cord 36, as described in the second embodiment. Accordingly, an operating space 32 for surgery can be thereby obtained between the target organ 22 and another organ 30.

The cord 36 in the top end side of the PTFE tube 34 may be inserted into the engaging hole 54 of the engaging member 52.

The intra-body-cavity insertion instrument according to the third embodiment of the invention is not limited to the endoscope 26 but may of course be an intra-body-cavity insertion instrument guide 10 including a lumen 16 as described in the first embodiment.

As has been described above, according to the third embodiment, an intra-body-cavity insertion instrument which is too thick to be inserted into the lumen 16 of the intra-body-cavity insertion instrument guide 10 or the forceps channel of the endoscope 26 can be grasped and guided by the biopsy forceps 28 inserted into the lumen 16 or the forceps channel. Accordingly, the target organ 22 can be supported more stably by such a thick intra-body-cavity insertion instrument.

### Fourth Embodiment

FIG. 5 is a view illustrating a structure of an intra-body-cavity insertion instrument guide system according to the fourth embodiment of the invention.

Also in the fourth embodiment, a PTFE tube 34 as described previously is used as an intra-body-cavity insertion instrument. In case of an endoscope 26 as an intra-body-cavity insertion instrument guide according to the fourth embodiment of the invention, a hard top end 44 is provided with an engaging member 56 for engaging and grasping the PTFE tube 34. The engaging member 56 is configured to be pivotally operated in a direction denoted by an arrow in the figure, and a grasp of the PTFE tube 34 is released by pivoting of the endoscope 26.

Therefore, the PTFE tube 34 can be guided simultaneously together with the endoscope 26 by inserting the endoscope 26 into a body cavity 20 while maintaining the grasp as described above. When the PTFE tube 34 reaches an opposite side over a target organ 22, the grasp by the engaging member 56 is then released, and the endoscope 26 is pulled out of the human body. The PTFE tube 34 is then left remain in the body cavity 20.

Thereafter, two ends of the PTFE tube 34 are engaged on ribs 18 or a frame not illustrated by the cord 36, as described in the second embodiment. Accordingly, an operating space 32 for surgery can be thereby obtained between the target organ 22 and another organ 30.

The intra-body-cavity insertion instrument guide according to the fourth embodiment of the invention is not limited to the endoscope 26 but may be of any type insofar as the intra-body-cavity insertion instrument includes an engaging member 56 pivoting of which can be operated from outside the human body.

As has been described above, according to the fourth embodiment, the intra-body-cavity insertion instrument can be grasped and guided, and accordingly, the target organ 22 can be supported more stably by a thick intra-body-cavity insertion instrument.

### Fifth Embodiment

FIG. 6 is a view illustrating a rectangular sheet 58 as an intra-body-cavity insertion instrument used in an intra-body-cavity insertion instrument guide system according to the fifth embodiment of the invention. Cords 60 as pulling members for pulling from inside a body cavity 20 to outside the human body are respectively attached to four corners of the sheet 58.

Such a sheet 58 as described above is wound about a top end section of a core pipe 62, as denoted by arrows in the figure, and is guided into the body cavity by an intra-body-cavity insertion instrument guide according to the fifth embodiment of the invention.

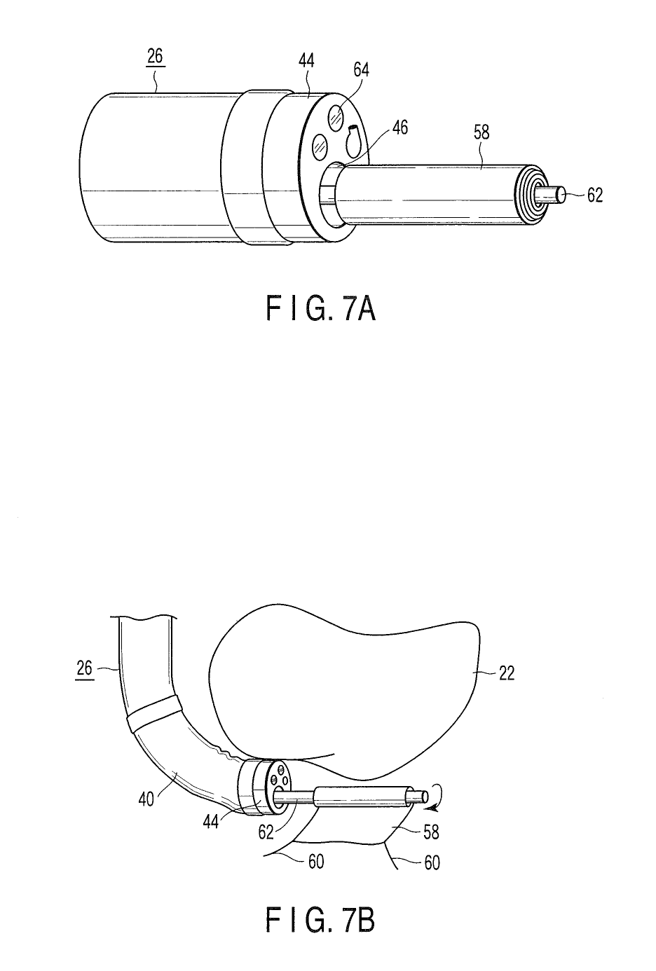

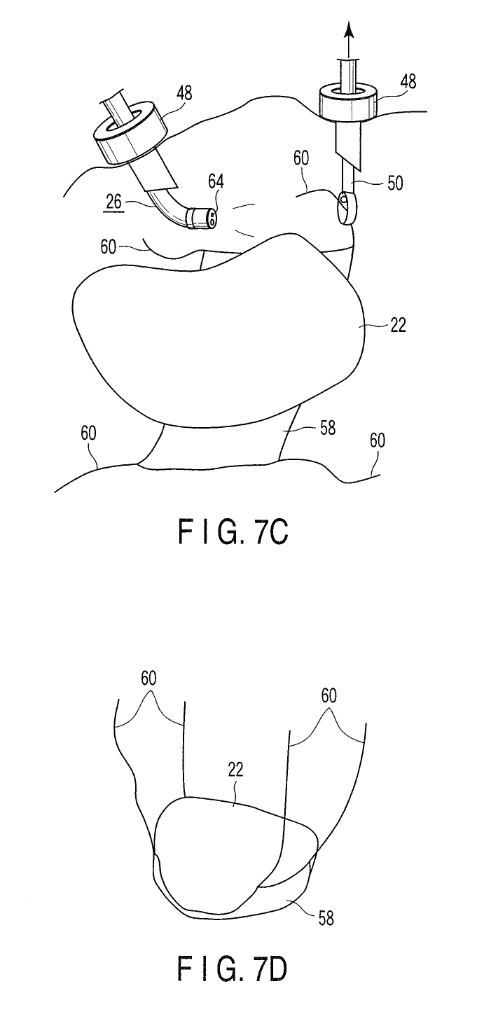

Specifically, an endoscope 26 as the intra-body-cavity insertion instrument guide according to the present embodiment is slipped in under the target organ 22, as in the second embodiment described previously. Further, when a hard top end 44 of the endoscope 26 reaches an opposite side over the target organ 22, the core pipe 62 with the aforementioned sheet 58 wound about is inserted into a forceps channel. As illustrated in FIG. 7A, the whole of a section where the sheet 58 is wound about is made to protrude from a tapered outlet port 46 of the hard top end 44. Thereafter, as illustrated in FIG. 7B, the core pipe 62 is rotated in a direction opposite to a winding direction of the sheet 58, to spread the sheet 58 wound about the core pipe 62. A state of spreading the sheet can be monitored through a camera 64 attached to a top end of the endoscope 26.

After the sheet 58 is completely spread out, the core pipe 62 is pulled out. As illustrated in FIG. 7C, a pair of forceps 50 is inserted into the body cavity 20 through a trocar 48 fixed to a side opposite to another trocar 48 in which the endoscope 26 is inserted. While monitoring through the camera 64 of the endoscope 26, the cords 60 attached to the sheet 58 are grasped with the forceps 50, and are pulled out of the human body through the trocars 48. As illustrated in FIG. 7D, the cords 60 attached to the four corners of the sheet 58 are pulled out of the human body by using the forceps 50 in this manner, and the cords 60 are engaged on ribs 18 or a frame not illustrated. The sheet 58 thereby lifts up the bottom face of the target organ 22 in the body cavity 20 against gravity, and separates the target organ 22 from another organ 30. Accordingly, an operating space 32 for surgery can be obtained between the target organ 22 and the another organ 30.

The intra-body-cavity insertion instrument according to the fifth embodiment of the invention is not limited to the endoscope 26 but may be of any type insofar as the intra-body-cavity insertion instrument can guide the core pipe 62 about which the sheet 58 is wound.

As has been described above, according to the fifth embodiment, the operating space 32 can be obtained by supporting the target organ 22 on a surface of the sheet.

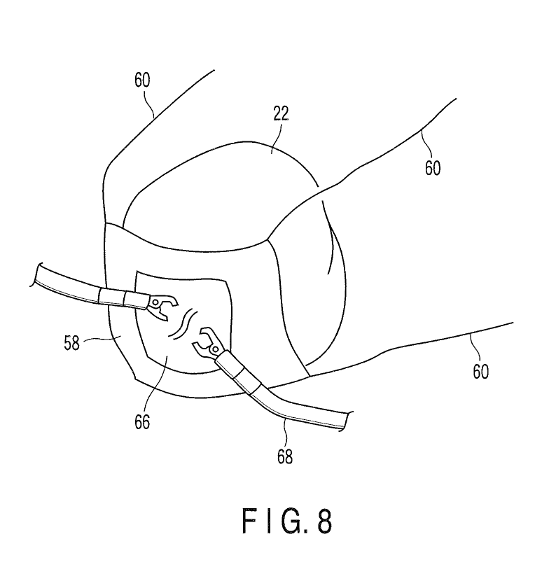

As illustrated in FIG. 8, a window 66 may be formed in the sheet 58, and a target portion to be operated on may then be operated on with a surgical instrument 68 through the window 66.

The present invention has been described above on the basis of embodiments. The invention, however, is not limited to the embodiments described above but may of course be variously modified or applied practically within the scope of the invention.

Additional Notes

Inventions as configured below can be extracted from the specific embodiments described above.

(1) An intra-body-cavity insertion instrument guide comprising:

an insertion section that is inserted into a body cavity; and

a guide section that is formed at the insertion section and guides an intra-body-cavity insertion instrument for obtaining a space for operating on an organ in the body cavity, to a side opposite to an insertion hole into which the insertion section is inserted, in relation to the organ.

### Corresponding Embodiments

The first to fifth embodiments correspond to embodiments relate to the intra-body-cavity insertion instrument guide described in (1). The body cavity 20 in these embodiments corresponds to the body cavity described above; the intra-body-cavity insertion sections 12 and 40 correspond to the insertion section described above; the target organ 22 corresponds to the organ described above; the cords 24, PTFE tube 34, and sheet 58 correspond to the intra-body-cavity insertion instrument described above; the lumen 16, forceps channel, biopsy forceps 28, and engaging member 56 correspond to the guide section described above; as well as the intra-body-cavity insertion instrument guide 10 and endoscope 26 correspond to the intra-body-cavity insertion instrument.

Operation and Effects

According to the intra-body-cavity insertion instrument guide described in (1), the intra-body-cavity insertion instrument can be guided to a deep portion in a body cavity. Therefore, a target organ at the deep portion in the body cavity can be separated from other organs, so that a space for surgery can be obtained.

(2) The intra-body-cavity insertion instrument guide described in (1), wherein the guide section guides the intra-body-cavity insertion instrument downwardly under the organ in a gravitational direction.

### Corresponding Embodiments

The first to fifth embodiments correspond to embodiments relate to the intra-body-cavity insertion instrument guide described in (2).

Operation and Effects

According to the intra-body-cavity insertion instrument guide described in (2), an organ is pressed against the intra-body-cavity insertion instrument due to gravity by positioning the intra-body-cavity insertion instrument at a lower position in the gravitational direction. Accordingly, not only an operating space can be obtained but also movement of an organ such as pulsation can be restricted.

(3) The intra-body-cavity insertion instrument described in (1), wherein the guide section includes a release section that releases and leaves the intra-body-cavity insertion instrument in the body cavity.

### Corresponding Embodiments

The second, fourth, and fifth embodiments correspond to embodiments relate to the intra-body-cavity insertion instrument guide described in (3). In these embodiments, the outlet port 46 and engaging member 56 correspond to the release section described above.

Operation and Effects

According to the intra-body-cavity insertion instrument guide described in (3), the guided intra-body-cavity insertion instrument is once left remain in the body cavity, and therefore, the position of the intra-body-cavity insertion instrument can further be finely adjusted.

(4) The intra-body-cavity insertion instrument guide described in (1), wherein the insertion section is a soft tube.

### Corresponding Embodiments

The first to fifth embodiments correspond to embodiments relate to the intra-body-cavity insertion instrument described in (4).

Operation and Effects

According to the intra-body-cavity insertion instrument guide described in (4), insertion along an organ can be achieved without damaging the organ.

(5) An intra-body-cavity insertion instrument guide system comprising:

an intra-body-cavity insertion instrument that obtains, in a body cavity, a space for operating on an organ in the body cavity; and

a guide that includes an insertion section inserted into the body cavity, and a guide section that is formed at the insertion section and guides the intra-body-cavity insertion instrument to a side opposite to an insertion hole into which the insertion section is inserted, in relation to the organ.

### Corresponding Embodiments

The first to fifth embodiments correspond to embodiments relate to the intra-body-cavity insertion instrument guide system described in (5). The body cavity 20 in these embodiments corresponds to the body cavity described above; the target organ 22 corresponds to the organ described above; the cords 24, PTFE tube 34, and sheet 58 correspond to the intra-body-cavity insertion instrument described above; the insertion sections 12 and 40 correspond to the insertion section described above; the lumen 16, forceps channel, biopsy forceps 28, and engaging member 56 correspond to the guide section described above; as well as the intra-body-cavity insertion instrument guide 10 and endoscope 26 correspond to the guide described above.

Operation and Effects

According to the intra-body-cavity insertion instrument guide system described in (5), the intra-body-cavity insertion instrument can be guided to a deep portion in a body cavity. Therefore, a target organ at a deep portion in the body cavity can be separated from other organs, so that a space for surgery can be obtained.

(6) The intra-body-cavity insertion instrument guide system described in (5), wherein the intra-body-cavity insertion instrument includes a pulling member for pulling from inside of the body cavity to outside of the body cavity.

### Corresponding Embodiments

The third and fifth embodiments correspond to embodiments relate to the intra-body-cavity insertion instrument guide system described in (6). The engaging member 52 and cords 60 in these embodiments correspond to the pulling member described above.

Operation and Effects

According to the intra-body-cavity insertion instrument guide system described in (6), the intra-body-cavity insertion instrument can be fixed to outside of the human body. Therefore, an obtained operating space can be steadily maintained.

What is claimed is:

1. An intra-body-cavity insertion instrument guide comprising:
an insertion section that is inserted into a body cavity; and
a guide section that is formed at the insertion section and guides an intra-body-cavity insertion instrument for obtaining a space for operating on an organ in the body cavity, to a side opposite to an insertion hole into which the insertion section is inserted, in relation to the organ.

2. The intra-body-cavity insertion instrument guide according to claim 1, wherein the guide section guides the intra-body-cavity insertion instrument downwardly under the organ in a gravitational direction.

3. The intra-body-cavity insertion instrument according to claim 1, wherein the guide section includes a release section that releases and leaves the intra-body-cavity insertion instrument in the body cavity.

4. The intra-body-cavity insertion instrument guide according to claim 1, wherein the insertion section is a soft tube.

5. An intra-body-cavity insertion instrument guide system comprising:
an intra-body-cavity insertion instrument that obtains, in a body cavity, a space for operating on an organ in the body cavity; and
a guide that includes an insertion section inserted into the body cavity, and a guide section that is formed at the insertion section and guides the intra-body-cavity insertion instrument to a side opposite to an insertion hole into which the insertion section is inserted, in relation to the organ.

6. The intra-body-cavity insertion instrument guide system according to claim 5, wherein the intra-body-cavity insertion instrument includes a pulling member for pulling from inside of the body cavity to outside of the body cavity.
